(19) United States
(12) Patent Application Publication
(10) Pub. No.: US 2024/0074711 A9
(43) Pub. Date: Mar. 7, 2024

(54) Big Data 911TM - Expedited EMT response after Vehicle Accidents or Other Emergencies

(71) Applicant: Mark Haley, McKinney, TX (US)

(72) Inventor: Mark Haley, McKinney, TX (US)

(21) Appl. No.: 17/302,264

(22) Filed: Apr. 29, 2021

Related U.S. Application Data
(63) Continuation-in-part of application No. 16/852,505, filed on Apr. 19, 2020.

(51) Int. Cl. A61B 5/00; G16H 10/60; H04M 3/22; H04W 4/029; H04W 4/16; H04W 4/44; H04W 4/90

(52) U.S. Cl. CPC A61B 5/747; G16H 10/60; H04M 3/2218; H04W 4/029; H04W 4/16; H04W 4/44; H04W 4/90; A61B 5/0022

(57) ABSTRACT

Device, system and method, for simplicity called Big Data 911™, which continuously sends GPS and other data to the cloud for storage and analysis, and Big Data automatically detects a vehicle crash and/or other emergency and immediately automatically calls 911 with crash information comprising the location, severity of the crash, etc. Victims, with their relevant medical records, are identified using onboard cameras and sensors or with medical information voluntarily linked via a phone app to the cloud. This medical data and GPS location ensures the optimum medical response during the crucial Golden Hour after the accident and that EMTs (Emergency Medical Technicians), police and other responders provide the optical medical treatment both en route to the hospital and once there. This includes AI or statistical information to expedite the ideal medical solution of the victim. This system could be used nationally or internationally as a person travels worldwide.

### TECHNICAL FIELD

Device, system and method to expedite response times after vehicle accidents or other emergencies by automatically initiating an expedited 911 computer call from a vehicle and/or a phone with data comprising GPS location, speed, etc. and the person's preapproved medical history all of which is continuously linked to the cloud before and/or after the crash. A vehicle's method and system comprising its onboard cameras, sensors and computers, and/or a phone's sensors, computer, etc. are continually linked to the cloud and automatically indicate when a crash and/or other emergency occurs using speed, acceleration and deceleration and other data thereby providing an inexpensive Big Data 911™. Using multiple sources of data when available offers redundancy to verify data and avoid unnecessary 911 calls. It expedites transportation to the hospital and ensures the optimum medical treatment customized for the victim during the crucial Golden Hour after the accident. This solution can be used nationally or internationally using Big Data in worldwide networks.

### BACKGROUND

Device, system and method which expedites Emergency Medical Technicians (EMTs), police, etc. response after Vehicle Accidents or Other Emergencies anywhere, such at home or in vehicle crashes, thereby ensuring the ideal medical care after accidents. There were 36,560 traffic deaths in 2018 and faster emergency team response times improves the chances for survival of hundreds or even thousands of accident victims each year. In 2018, 10% of fatal crashes were reported in over ten minutes, This-identifies technologies and economic incentives so that drivers permit sharing crash data automatically during the seconds leading to an accident. The GPS data, medical histories, and other data is continuously connected and to and stored in the cloud and in a millisecond once a crash and/or other emergency is detected an automatic call is made to the 911 network either with a text to voice link or with all the relevant info displayed on the 911 call center screens,. The vehicle and/or phone send GPS data in a continuous link to the enhanced 911 cloud network. Using standard GPS data, with as little as 100 characters comprising the latitude, longitude, elevation and with as little as once per second, this pinpoints the location and speed of the tracker and also confirms the type of vehicle and when a crash occurs. A vehicle automatically identifies its type of vehicle. However, a smart phone can only determine the type of vehicle using GPS data cross checked with historical data to confirm when a person is in a car, train, plane, even when the person jumps out of the plane and skydives. The author used this technique to track hundreds of trains, planes, cars and skydives and in accident investigations. While larger GPS databases provides more precision, even a small database of GPS data identifies the type of vehicle. The method and system uses one or more sensors, including GPS data, on vehicles and/or phones to automatically make 911 calls when needed (FIG. 1-6). However, this method and system has enough information using only the GPS data from a smart cell-phone to make a 911 call (FIG. 7).

In 2018, only 10% of U.S. drivers permitted sharing telematic vehicle data, the key info on the vehicle's speed, location, acceleration, etc. However, with larger automobile insurance discounts, more privacy protections and better technology, more drivers could be enticed to share this data thereby expediting response times and improving the chances for survival of accident victims, The European Union is far ahead of the US since after Apr. 2018 all European Car Makers have been required to include eCall, an automated emergency call technology. Big Data is the rapid analyzes of massive amounts of data to solve preciously unsolvable problems. Big Data 911™ harnesses data from the vehicle and/or phone, the cloud plus the key info which is voluntarily disclosed by passengers linked to the cloud and data from a vehicle's cameras and sensors which monitor its speed, location, and identifies the driver using onboard cameras and facial recognition software (FIGS. 1-3). A vehicle generates terabytes of data per day and millions of vehicles must be tracked. Key crash data is automatically sent to 911 even from remote locations, or at night, where there aren't manual 911 calls. These robotic calls identify the location and severity accidents, the driver's medical history, etc. helping save victims in the Golden Hour after crashes when the chances of survival are the highest. Also, by constantly monitoring vehicles, even when there is no data, such as failures of onboard sensors or loss of communications, this indicates potential problems or accidents (FIG. 4). This system work nationally and internationally (FIG. 5).

Big Data 911™ expedites the response times of first responders after traffic accidents thereby potentially saving lives or reducing injuries. On crowded urban U.S. roads many people immediately call 911 after an accident. Some cars even automatically send out distress calls. However late at night and in remote areas, the accident may not be reported for hours. Enhanced Big Data and the cloud automatically report those accidents during the seconds leading up to a crash, rather than 10 minutes after the accident.

Today's newest ear sensors collect terabytes of data each day some of which is used to autonomously drive the car or even brake in potential accidents. This device, system and method invention focuses on ensuring that key parts of this data is sent to the cloud to alert first responders immediately after an accident.

### “Golden Hour”—It's Crucial to Expedite Treatment Within an Hour to Save Lives

Per the American College of Surgeons, the idea of the “Golden Hour” highlights the crucial need to successfully treat a patient in the first 60 minutes after a major injury such as an automobile crash or a gunshot wound. The method of treating trauma is call the “Advanced Trauma Life Support (ATLS)”. It was developed in 1976 based on experiences treating those seriously injured during the Vietnam War and in dangerous U.S. cities.

### BRIEF DESCRIPTION OF THE DRAWINGS

FIG. 1 Big Data and 911 Expedites Emergency Medical Care (Overview)

FIG. 2 Sample Sensors/Cameras in 2021 Autonomous Vehicles

FIG. 3 eCall is a good 1^{st} Step the 2^{nd} step is Big Data and Cloud Tracking

FIG. 4 Big Data and Cloud Monitors Drivers even with no Cell Network

FIG. 5 GPS/other Data from Millions of Vehicles/Phones sent to the Cloud Optimizing Emergency Response

FIG. 6 Big Data and Cloud Network Issues—Key Redundancy of Communications

FIG. 7 Emergency Network Tracking using only GPS and/or GPS and other Data

### DETAILED DESCRIPTION

This device, system and method using Big Data and the Cloud offers an integrated method of responding to vehicle accidents or general 911 calls so that EMTs, police, etc. know the medical background of the victims(s) and provide appropriate treatment immediately and during the transport to the hospital. The goal is to expedite moving the victim to the hospital and to provide the ideal medical treatment in the Golden Hour after an accident to ensure the best medical outcome. For vehicle accidents, a device, ideally mandated by the government similar to the mandate for smoke alarms, would automatically send out a distress call to 911 as the vehicle data indicated an impeding crash. This call would be just prior to impact in case it is a massive wreck which disables all communications and/or on-board computers. The information includes the identity of the driver using cameras plus facial or voice recognition software which monitors the driver as part of the autonomous driving system for the latest vehicles. Alternatively, in case the system fails to identify the passenger(s), and due to privacy concerns, an option to voluntarily provide medical records into the vehicle's computers or phone provides the medical history the passenger want to disclose for accidents.

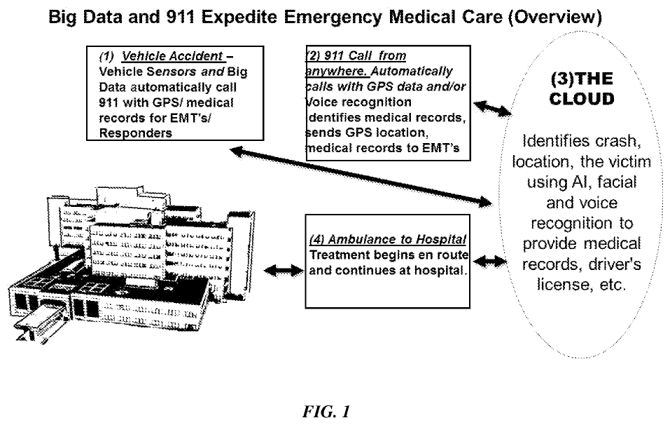

In FIG. 1, Block 1, a vehicle's computers and software, automatically call 911 with the GPS data and medical records for EMT's Responders. If it's a 911 call from a phone it also automatically sends GPS data and/or Voice recognition identifies medical records and sends the GPS location and medical records to EMT's.

Using voice or facial recognition software calls or voluntarily uploaded data plus cross checking with cloud data in either a vehicle accident or a 911 call from home, the location, the identity and medical background of the victim is known. Therefore, the EMTs or other responders can customize the treatment of the patient in route to the hospital. Block 3—using facial or voice recognition software or caller ID info, or voluntary disclosed information from a phone app linked to the vehicle's computers, the victim's medical records are available from Big Data in the cloud with Artificial Intelligence (AI) constantly reviewing the appropriate medical issues and treatments. While being transported to the hospital, Block 4, this system has identified the patient with related medical histories so the EMTs can provide initial customized treatment which would continue at the hospital.

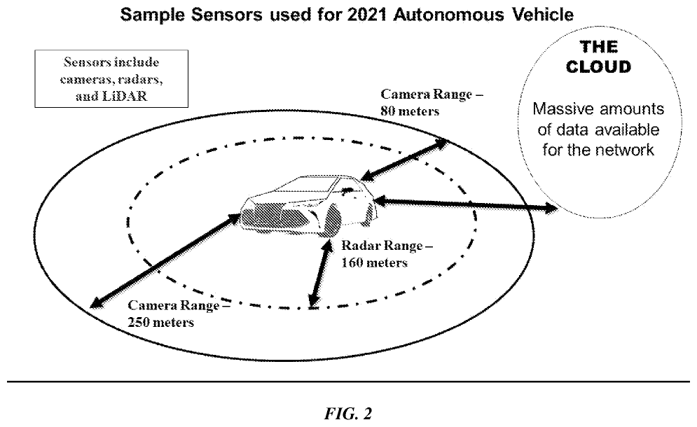

FIG. 2 shows that autonomous vehicles use a large number of cameras, radars and LiDAR to map the world around the vehicle and thereby plan the optimal, safe drive. In 2021, the industry leader Tesla used 8 cameras with 360 degrees of view with a range of up to 250 meters in front and 50 meters behind, while ultrasonic sensors enhanced the mapping around the car and radar even permitted seeing ahead even during rain, or in the fog or with dust.

However, internal cameras used to monitor the driver's condition have created controversy. These internal cameras record and upload to the cloud the driver's actions including prior to a crash. While the user can opt-out of this option, this is creating privacy concerns. To address these privacy concerns and due to the limitations of facial and voice recognition software, the passengers in a vehicle could voluntarily disclose medical and other information to the vehicle's computers manually or via an automatic phone app when they enter the vehicle.

### Automated Emergency Calls are Mandated in the EU and Should be Required in the U.S.

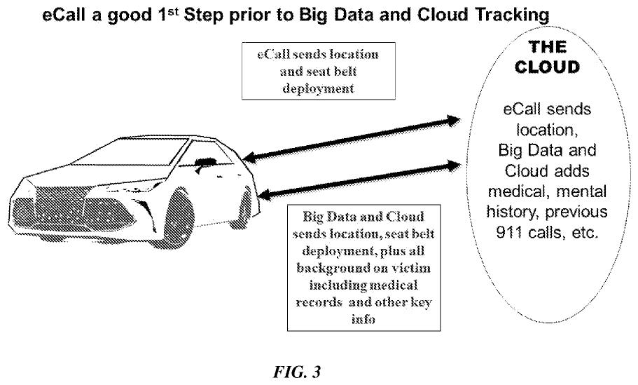

FIG. 3—Telematics is a tracking device in vehicles which sends key information such as location, speed and harsh braking or acceleration. Up to 10 percent of drivers in the US use this technology since insurance companies offer discounts for sharing this data. However, in 2021 the European Union was far ahead of the US since after Apr. 2018 all European Car Makers have been required to include eCall, an automated emergency call technology. When a crash is occurring eCall automatically sends the location and also indicates if the airbags were deployed. However, using GPS data and other sensor information, Big Data with facial and voice recognition software identifies the driver's ID, medical history and all relevant information needed to treat the victim.

### Track Me—Option in Sporadic Cell Phone Coverage Areas

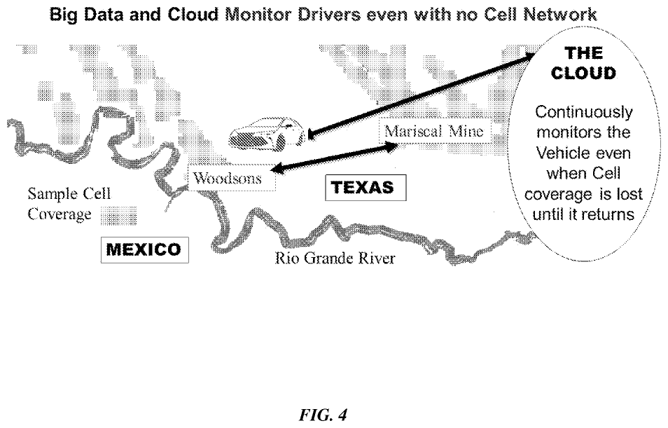

FIG. 4—One of the most remote areas in the lower 48 states is the Frank Church-River of No Return Wilderness in Idaho which has 2.37 million acres. It almost limited if any cell phone coverage. For example, Dixie Idaho (2019 population, 3,237) is near this area had limited cell coverage (2019). Large areas in Alaska have almost no coverage. The drive from Woodsons, Texas which is inside Big Bend National Park, to the Mariscal Mine, which is also in Big Bend, illustrates the problem and solution. This 11-mile drive takes roughly 55 minutes has sporadic cell coverage. In Big Bend, wilderness areas and many National Parks, there should be the telematics option to turn on an optional Track Me where the system monitors the status of those vehicles which have lost cell coverage. After a pre-determined time, it would make an automatic phone call to confirm that the driver was OK. And then after a specified time even a 911 call could be made. This option could also be valuable when there was equipment failure on-board the vehicle or a breakdown of part of a cell network.

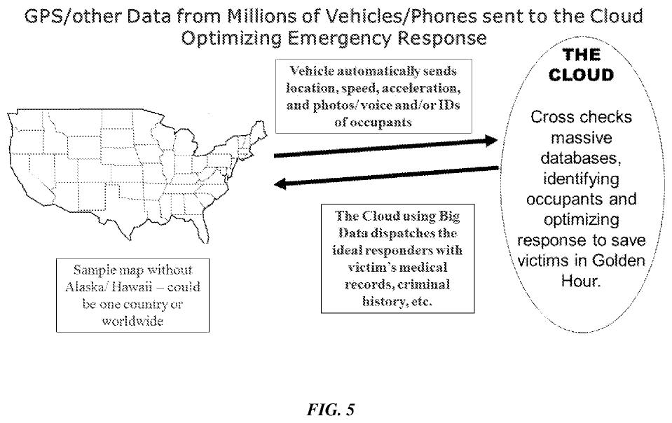

FIG. 5—The system sends info on millions of vehicles to the cloud optimizing the emergency response. The vehicle and/or phone app automatically sends its GPS location, speed, acceleration, and photos/voice and/or IDs of occupants plus any voluntary medical ID provided by the occupant. The phone can also provide redundant GPS data which confirms the crash to the cloud in order to minimize the number of incorrect 911 calls. The cloud cross checks massive databases, including crash data and vehicles' crash data histories identifying occupants and optimizing response to save victims in Golden Hour

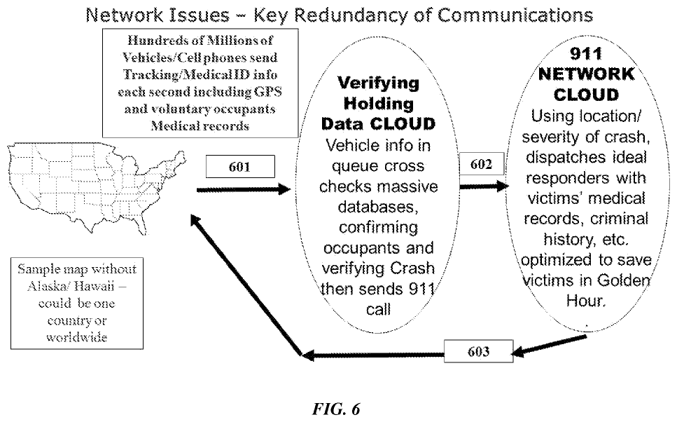

FIG. 6. Network Issues—Key Redundancy of Communications. FIG. 6 provides additional details for FIG. 5. FIG. 6. 601 shows GPS data can be sent in small packets of only 100 characters using either a smart phone or computers on the vehicles. While additional data could complement the system, the GPS data is sufficient to confirm the location of the crash and its severity including the crash speed, etc. Since the medical records only change at most every 10 minutes, assuming people entering or exiting the vehicle, the average size of the key medical data is less than 40 characters per second. However, this is illustrative and the number of bytes needed and the time interval could be adjusted based on needs to expedite EMTs response times and given the issues of network storage and bandwidth. Over time, with the massive amounts of vehicle information, more data beyond GPS and medical information could be sent on the network if these improved emergency response times.

In 2021, per the US Department of Transportation there were 276 million vehicles in the U.S. including 156 million trucks, 108 million cars, 8.5 million motorcycles, and 575 thousand buses. Statista data estimated that the USA has 280 million smartphone users in 2020. Redundancy in communications is crucial due to equipment malfunction and network failures. For example if a person carried a cell phone on a motorcycle and dropped it that would indicate a crash, however if there were an onboard computer on the motorcycle which confirmed that the motorcycle did not crash that would avoid an unnecessary 911 call.

FIG. 6 shows that even with 276 million vehicles and 280 smart phones sending 100 bytes of GPS and medical data per second, this is very low bandwidth for any device a network, such as a 5G network. With millions of vehicles and phones, even small data packets sent to the cloud create hundreds of Gigabyte of data each second or Terabytes of data hour. However, the only data that needs to be stored is the crash data and emergency data and due to privacy concerns all other unrelated data would not be stored and/or needed. As a result the data storage requirements in the cloud are relatively small. FIG. 6 602 shows the data sent to a queue which uses AI (artificial intelligence and/or statistics), to verify that a crash has occurred, ideally with multiple sources of GPS data, i.e. from both the vehicle's computer link or ideally multiple links plus the option of the call phone link as backup.

Finally, the 911 NETWORK CLOUD—FIG. 6 603 uses the GPS location and the severity of crash, and dispatches ideal responders with victims' medical records, etc. optimized to save victims in Golden Hour.

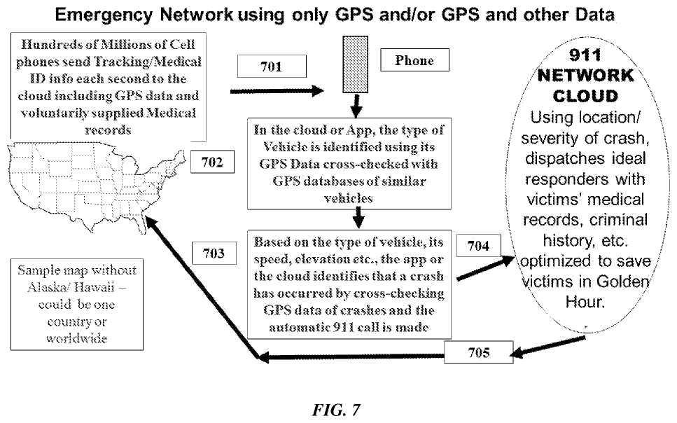

FIG v. 7 shows the system works using only GPS data from a vehicle and/or cell phones FIG. 7 701. In 2022, most vehicles lacked the advanced electronics to automatically send GPS data therefore the only information provided might be cell phone data. A person can make a manual 911 call however the best solution is this system where a cell phone app automatically calls 911 with the GPS location data comprising the speed and severity of the crash. An automatic 911 call would be made even if the occupant of the vehicle was unable to call. With only GPS data, the system cross-checks historical databases GPS profiles of similar vehicle to confirm the type of vehicle, i.e. a car, or train or plane since, the vehicle GPS data and crash data of each vehicle is different. Therefore the GPS data confirms the type of vehicle FIG. 7 702 and then using the speed and other parameters of that vehicle determines if a crash has occurred FIG. 7 703. The system could use AI and/or statistical data to minimize unnecessary 911 calls. The first priority would be to respond to calls which had redundancy with multiple sensors FIG. 7 704. The network would give a lower priority to 911 calls generated from only cell phone generated calls to avoid overloading the 911 system FIG. 7 705.

FIG. 7 comprises continuous coverage of GPS tracking, however due to privacy concerns, the users could opt in to tracking, such as automatically when the person enters a vehicle, or for children who leave their home. Ultimately, based on Big Data collected overtime, AI could determine to make a 911 call and the system could be adjusted to limit tracking to only vehicles if too many incorrect 911 calls were made.

What is claimed is:

1. A method comprising: continuously sending GPS and other data to the cloud from a vehicle's sensors and cameras and/or from a smartphone's sensors, to automatically detect a vehicle crash and/or other emergency using GPS and other data, cross-checking with historical crash data and immediately automatically calling 911 with all crash information, and sending the victim's medical records and caller ID which are collected using onboard cameras to expedite the response time by EMTs (Emergency Medical Technicians) for vehicle crashes and other emergencies focusing on the customized treatment of the victim during the crucial Golden hour or beyond of an incident; monitoring, via a call-taking computing device, a call from a caller reporting an incident of a given incident type; using information including caller ID, voice and facial recognition, prior 911 calls to identify the victim and provide the appropriate medical treatment en route to the hospital and then continuing on after arrival at the hospital or medical facility.

2. The method of claim 1 further comprising: using the vehicle's video and/or audio, or voluntarily uploaded app data confirming the caller's ID to retrieve prior medical history, police reports including jail records and mental history which indicates police may be needed, and comprising all relevant data in order to immediately begin the appropriate care and response associated with the incident-type profile for the call; and using Advanced Automatic Collision Notification (AACN) and the injury severity score (ISS) to improve post-crash medical care using Big Data in the cloud to monitor millions of vehicles and/or individuals anywhere in the world to provide the optimal treatment after accidents.

3. A system comprising: a communication unit, a GPS unit, and a link to a vehicle's on-board sensors to continually monitor from the cloud and store the vehicle's location, acceleration, deceleration, elevation, speed and the status of the vehicle's systems and cross checking historical crash data to determine if an emergency condition exists including a crash, fire or other emergency and to then send a 911 distress call to request the dispatch of EMT and/or police to the incident with all related medical information and other records of the potentially injured party.

4. A system wherein the victims are identified using vehicle's cameras and facial and/or voice recognition software and this info is cross-checked with the cloud and the option to voluntarily provide medical records linked via a phone to the vehicle's computers to generate and update the accident victim's profiles based on one or more of data obtained in association with the call and the historical data, the historical data further comprising one or more of: previous call data; other call data; computer aided dispatch data; police records; incident resolution data; evidence data; jail data; social media data; medical records; security records; and customer records.

5. The method of claim 1, further comprising, tracking all vehicles which have lost vehicle and/or cell contact with the cloud and after a time period selected by the driver, automatically calling the driver confirming their status and/or calling 911 if the loss of cell coverage exceeds a preset time limit.

6. The method of claim 1, further comprising a smart phone app and/or software linked to the cloud which continuously sends the phone's GPS data to track the vehicle and/or person and confirm when crashes occur using acceleration and deceleration data cross-checked with databases of prior crashes of similar vehicles thereby providing the cloud accurate crash and/or other emergency notification and avoiding incorrect 911 calls.

7. The method of claim 1, further comprising tracking via GPS data and consensually shared medical records from phone apps and/or vehicle sensors expediting medical help from responders after crashes or other emergencies where the redundancy of the tracking devices ensures that automatic calls to the 911 network occur in an emergency and not due to lost and/or corrupt GPS and other data and/or equipment malfunctions.

8. A system comprising: vehicle and/or smartphone sensors and cameras sending GPS and other data tracking the occupants and vehicle and using GPS location, speed and other data confirming the type of vehicle tracked and when the vehicle crashes and/or other emergency occurs and storing this data in the cloud to continuously improve the analysis of vehicle crash data to reduce the chances of incorrect 911 calls and the system makes an automatic call to the 911 network and cloud requesting emergency responders.
